(19) United States
(12) Patent Application Publication
(10) Pub. No.: US 2009/0148450 A1
(43) Pub. Date: Jun. 11, 2009

(54) ATF4 As A Therapeutic Target In Alzheimers Disease And Other Neurological Disorders

(76) Inventors: Lloyd Greene, Larchmont, NY (US); Michael Shelanski, Brooklyn, NY (US); Conrad Leung, New York, NY (US); Ottavio Valerio Vitolo, Cambridge, MA (US)

Correspondence Address: BAKER BOTTS L.L.P., 30 ROCKEFELLER PLAZA, 44TH FLOOR, NEW YORK, NY, 10112-4498, US

(21) Appl. No.: 12/209,713

(22) Filed: Sep. 12, 2008

Related U.S. Application Data
(63) Continuation of application No. PCT/US07/06579, filed on Mar. 15, 2007.
(60) Provisional application No. 60/783,284, filed on Mar. 17, 2006.

(51) Int. Cl. A61K 39/395; A61K 31/7052; G01N 33/68; G01N 33/50; C07H 21/00; C07K 16/00; C12Q 1/02; C12Q 1/68; A61P 25/00

(52) U.S. Cl. 424/135.1; 514/44; 436/86; 436/94; 536/23.1; 530/387.9; 435/29; 435/6

(57) ABSTRACT

The present invention relates to methods and compositions for treating Alzheimer's Disease and other neurological disorders by inhibiting expression and/or activity of ATF4. It further provides for diagnostic methods and reagents as well as assays to identify agents for the treatment of Alzheimer's Disease and other ATF4-related conditions.

### PRIORITY CLAIM

This application claims priority to U.S. Provisional Application No. 60/783,284, filed Mar. 17, 2006, the contents of which is incorporated herein in its entirety.

### 1. INTRODUCTION

The present invention relates to methods and compositions for treating Alzheimer's Disease and other neurological disorders by inhibiting expression and/or activity of ATF4. It further provides for diagnostic methods and reagents as well as assays to identify agents for the treatment of Alzheimer's Disease and other ATF4-related conditions.

### 2. BACKGROUND OF THE INVENTION

### 2.1 Long-Term Memory Formation

Memory and its underlying synaptic plasticity have temporal stages that rely on different molecular events. A short-term memory (STM) process lasting minutes, and a long-term memory (LTM) process lasting hours, days or years constitute the two major categories of memory. During the production of STM, preexisting proteins are temporarily modified, establishing memory that will only be retained for minutes. During LTM, signal transduction pathways result in de novo gene expression and protein translation. The establishment of these new proteins result in memory that will persist for long periods of time (Goelet, P. et al., 1986, Nature 322:419-422; Montarolo, P. et al., 1986, Molecular Aspects of Neurobiology, Montalcini, P. et al. eds. Berlin: Springer-verlag, Pg. 1-14).

Spatial and contextual conditioning memories have been the most widely studied forms of LTM in mammals. The hippocampus has been shown to be an important region for LTM generation in spatial LTM (Morris, R. et al., 1982, Nature 297:681-683), while both the hippocampus and amygdala are important for contextual conditioning LTM (Anagnostaras, S. et al., 2001, Hippocampus 11:8-17; Phillips, R. et al., 1994, Learn Mem. 1:34-44; Frankland, P. et al., 1998, Behav Neurosci. 112:863-874). The process of LTM formation requires the binding of the transcription factor cAMP Response Element Binding protein (CREB) to cAMP Response Element (CRE) containing genes in response to post-synaptic stimulation. Binding to the CRE site initiates the transcription of genes necessary for the establishment of LTM. The activation of CREB can occur through multiple signal transduction pathways, all involving protein activation through kinase dependent phosphorylation. For example, activation of the glutamate NMDA receptor allows entrance of calcium into the cell, resulting in the sequential activation of Ca²⁺/calmodulin (CaM), CaM-kinases (CaMKs), and finally CREB (Lonze, B. et al., 2002, Neuron 35:605-623; Soderling, T., 1999, Trends Biochem Sci. 24:232-236). One of the CaMKs that phosphorylates and activates CREB directly, CaMKIV, is important for both spatial and contextual LTM formation in the hippocampus. Kang et al. (2001, Cell 106:771-783) showed that mice expressing a forebrain-restricted dominant-negative CaMKIV (dnCaMKIV) were impaired in both spatial and contextual LTM formation, while Ho et al. (2000, J Neurosci. 20:6459-6472) found that only contextual LTM was affected in CaMKIV null mice.

Mechanisms other than calcium-dependent signal transduction pathways may result in CREB activation and CRE gene expression. Stimulation of G protein-coupled receptors, such as PAC1, can initiate a cAMP dependent signal transduction cascade resulting in the activation of protein kinase A (PKA), and subsequent CREB activation (Abel, T. et al., 1997, Cell 88:615-626). Furthermore, receptor tyrosine kinase-dependent activation of the MAP-kinase signal transduction pathway may also result in CREB activation and de novo gene expression necessary for generating LTM.

### 2.2 ATF-4

In addition to activators of CREB dependent LTM formation, memory suppressors act in parallel to set the threshold for long-term plasticity and LTM generation (Abel and Kandel, 1998, Brain Res. Brain Res. Rev. 26: 360-378). For instance, calsineurin, a cytoplasmic phosphatase, regulates the formation of LTM by gating the calcium/PKA signal transduction cascade. ATF4 (Activating Transcription Factor 4, also known as CREB-2 and tax-responsive enhancer element B67 (TAXREB67)), another LTM suppressor, interacts with C/EBP (CCAAT/Enhancer Binding Protein) family members to bind CRE sites and suppresses LTM dependent gene expression. ATF4 contains a leucine zipper region that is involved in protein-protein interactions, as well as a stretch of basic C-terminus amino acids that bind DNA. Like the LTM activators, ATF4 is turned on in response to cAMP activation during the onset of LTM initiation. (Chen, A. et al., 2003, Neuron 39(4):655-69; Gachon, F. et al., 2001, FEBS Lett. 502(1-2):57-62). Helm et al. (June 2005, Neuropharmacology 48(7):956-64) provide evidence that the GABA_{B} receptor antagonist SGS742 may enhance LTM generation by inhibiting the action, or reducing the expression, of ATF4.

In addition, ATF4 has also been postulated to play a role in the response of cells to endoplasmic reticular (ER) stress and in cell death (see Rutkowski, D. and Kaufman, R., 2003, Dev. Cell. 4(4):442-444) In response to ER stress (which may occur in response to toxic factors such as ischemia, and which can be manifested by improper protein processing and perturbed calcium homeostasis), a cell may develop an Unfolded Protein Response (UPR) in which expression of protein chaperones is increased and/or mRNA translation is decreased. If the level of unfolded protein reaches a critical threshold, cell death ensues, mediated by ATF4 and ATF6 and activation of the JNK/AP-1/Gadd152 pathway (Kim, R. et al., 2006, Apoptosis 11(1):5-13). According to Katayama, T. et al., 2004, J. Chem. Neuroanat. 28(1):67-78), familial Alzheimer's Disease-linked presenilin 1 downregulates the UPR and creates vulnerability to ER stress. However, Hoozemans, J. et al., 2005, Acta Neuropathol. (Berl) 110(2):165-172 reports that the UPR is activated in the context of Alzheimer's Disease; they postulate that this effect, which may initially be protective, might eventually lead to neurodegeneration.

### 2.3 Alzheimer's Disease

Alzheimer's Disease (AD) is characterized by a progressive decline in cognitive functions. Neuropathologies of the disease include the accumulation of tangles, β-amyloid containing plaques, dystrophic neurites, and loss of synapses and neurons (Selkoe, D. et al., 1999, Alzheimer's Disease, Ed2. Terry R. et al., eds. pg. 293-310. Philadelphia: Lippincott, Williams and Wilkins), but these pathologies are preceded by deficits in spatial and LTM generation (Vitolo, O. et al., 2002, Proc Natl Acad Sci USA. 99(20):13217-21. Epub 2002 Sep. 20).

AD exists as sporadic as well as heritable familial forms. While the sporadic version is more prevalent, study of familial AD may provide insight into sporadic AD since pathologies of both are similar. Familial AD results from mutations in the presenilin genes, an essential component of the γ-secretase enzyme complex; or amyloid precursor proteins, a substrate of γ-secretase and the precursor of β-amyloid. These mutations result in the accumulation of β-amyloid protein plaques in the brains of affected individuals (for review see Beglopoulos, V. et al., 2006, Trends Pharmacol Sci. 27(1):33-40. Epub 2005 Dec. 7; McCarthy, J., 2005, Biochem Soc Trans. 33(Pt 4):568-72; and Rosenberg, R., 2005, Arch Gen Psychiatry 62(11):1186-92).

As AD progresses, and β-amyloid continues to accumulate, patients exhibit impairment of short term memory, although memories formed years prior to disease onset may persist. The ability to generate new memories, including LTM, is gradually lost. Inhibiting CREB activation and the subsequent decrease in CRE gene expression is one mechanism for inhibiting LTM generation. Molecular evidence for β-amyloid's role in LTM inhibition has been shown by its ability to downregulate CREB activity. (Puzzo, D. et al., 2005, J Neurosci. 25(29):6887-97). Because CREB activation occurs through multi-step signal transduction pathways, there are numerous stages where an inhibitory pathway may act to block. the activation of CREB. Wang et al. have shown that β-amyloid can inhibit LTM through the stimulation of the kinases JNK, Cdk5, and p38 MAPK after the activation of both the β-amyloid receptor(s) and the glutamate receptor mGluR5 (Wang, Q. et al, 2004, J Neurosci. 24(13):3370-8). Dineley et al. have also shown that β-amyloid can inhibit CREB activation by reducing its state of phosphorylation. β-amyloid couples to the mitogen-activated protein kinase (MAPK) cascade via alpha7 nicotinic acetylcholine receptors (nAChRs). In vivo elevation of β-amyloid, such as that exhibited during AD, leads to the upregulation of alpha7 nAChR protein. alpha7 nAChR upregulation occurs concomitantly with the downregulation of the 42 kDa isoform of extracellular signal-regulated kinase (ERK2) MAPK in hippocampi of aged animals. The phosphorylation state of CREB protein, a transcriptional mediator of long-term potentiation and a downstream target of the ERK MAPK cascade, was also reduced.

Furthermore, Vitolo et al. demonstrate that β-amyloid treatment of cultured hippocampal neurons may inhibit CREB phosphorylation through the inactivation of protein kinase A (PKA) and promote the persistence of its regulatory subunit PKAIIalpha. Consistent with this, CREB phosphorylation in response to glutamate is decreased, and CRE gene expression is not induced (Vitolo, O. et al., 2002, Proc Natl Acad Sci USA. 99(20):13217-21, Epub 2002 Sep. 20).

Increases in β-amyloid perturb NMDA receptor function as well as the CREB signal transduction pathway. Snyder et al. found that β-amyloid promoted endocytosis of NMDA receptors in cortical neurons. In addition, neurons from a genetic mouse model of Alzheimer's disease expressed reduced amounts of surface NMDA receptors. Reducing β-amyloid by treating neurons with a γ-secretase inhibitor restored surface expression of NMDA receptors. Consistent with these data, β-amyloid application produced a rapid and persistent depression of NMDA-evoked currents in cortical neurons (Snyder E M, et al., Nat Neurosci. 2005 August; 8(8):1051-8. Epub 2005 Jul. 17). By reducing the activity level of NMDA receptors, less calcium would enter the cell, producing less CaM and CaMK activation, resulting in a reduced level of phosphorylated active CREB. Without the proper active phosphorylated state, CREB is unable to initiate the transcription of CRE genes, resulting in the loss of LTM generation.

### 3. SUMMARY OF THE INVENTION

The present invention relates to methods and compositions for treating and diagnosing Alzheimer's Disease and other ATF4-associated diseases. It is based, at least in part, on the discovery that ATF4 is elevated (i) in the context of overexpressed amyloid beta peptide; (ii) in a murine model of Alzheimer's Disease; and (iii) in human Alzheimer's patients, where the level of expression appears to correlate with severity of disease. It is further based on the discovery of siRNAs which inhibit ATF4 expression.

In non-limiting embodiments, the present invention provides for methods of treating Alzheimer's Disease or other neurological disorders associated with increased ATF4 expression, comprising administering, to a subject in need of such treatment, an agent which inhibits ATF4. In this regards, the present invention provides examples of siRNAs which have been shown to be effective in inhibiting ATF4 expression.

In further non-limiting embodiments, the present invention provides for methods of diagnosing Alzheimer's Disease or other neurological disorders associated with increased ATF4 by detecting elevated levels of ATF4 in a sample collected from a patient. In non-limiting embodiments, ATF4 protein expression may be detected and measured using antibodies directed to ATF4.

In still further embodiments, the present invention provides for drug discovery methods that identify agents that may be useful in the treatment of Alzheimer's Disease or other neurological disorders associated with increased ATF4 expression by screening for agents that inhibit ATF4 expression.

### 4. BRIEF DESCRIPTION OF THE FIGURES

FIG. 1. Relative abundance of ATF4 levels in hippocampi of J20 APP transgenic versus control mice as a function of age.

FIG. 2A-B. Immunostaining with anti-ATF4 antiserum of hippocampus from either (A) a J-20 APP transgenic mouse; or (B) a wild-type mouse.

FIG. 3. Relative expression of ATF4 transcripts (as determined by quantitative real-time PCR) in human entorhinal cortex as a function of various stages of AD and normal (NL) condition. *P<0.05.

FIG. 4A-E. Immunostaining for ATF4 in entorhinal cortex of post-mortem control and AD brains. (A) control (NL) brains; (B) eAD=early stage AD brains; (C) mAD=medium stage AD brains; (D) sAD=severe stage AD brains. Staining was achieved with antiserum #21 and the micrograph was taken under low magnification (400×). (E) Graph showing proportion of neurons showing intense nuclear staining for ATF4 in the post-mortem hippocampi of patients without AD (“NL”) and with AD (“mAD”).

FIG. 5. Immunostaining for ATF4 in hippocampal CA1 region of post-mortem AD brain. Some neurons showed intense nuclear staining of ATF4 (arrow). The picture was taken under high magnification (1000×).

FIG. 6A-C. (A) nucleotide sequences of siRNAs, siRNA1 (SEQ ID NO:5) and siRNA2 (SEQ ID NO:6). (B) SW13 cells were transfected with ATF4 siRNA1 or control siRNA. (C) SW13 cells were transfected with ATF4 siRNA2. For both (B) and (C), cell lysates were collected 24 hours after transfection and subjected to Western blotting with anti-ATF4 #18 antibody and anti-β-tubulin antibody. β-tubulin was used as a loading control.

FIG. 7. pCRE-d2EGFP vector. Unique restriction sites are in bold.

FIG. 8A-B. Western blots of SW13 cells either transiently transfected with pFLAG-DATF4 or mock transfected, probed with (A) #18 antibody or (B) #21 antibody.

FIG. 9A-B. Western blots using anti-ATF4 antibody #18 as probe: (A) SW13 cells treated with tunicamycin; or (B) human brain lysate.

FIG. 10. The percentage of PC12 cells with neurites over 5 days of NGF treatment, where the PC12 cells are engineered to express either eGFP (a marker for engineered cells) alone (“control”) or eGFP+ATF4-specific interfering RNA (“ATF4 shRNA”).

FIG. 11 The average length of neurites in cells as prepared for FIG. 10, after 5 days of NGF treatment.

### 5. DETAILED DESCRIPTION OF THE INVENTION

For clarity, and not by way of limitation, the detailed description of the invention is divided into the following subsections:
(i) anti-ATF4 agents;
(ii) methods of diagnosing Alzheimer's Disease or other ATF4-related disorders;
(iii) methods of treating Alzheimer's Disease or other ATF-4 related disorders; and
(iv) drug discovery methods.

Alzheimer's Disease is alternatively referred to as “AD” herein.

### 5.1 Anti-ATF4 Agents

The present invention provides for agents that antagonize ATF4 action, referred to herein as “anti-ATF4 agents”. Non-limiting examples of anti-ATF4 agents include agents that inhibit transcription or translation of ATF4 as well as agents that inhibit binding of ATF4 to its target DNA, including competitive as well as non-competitive inhibitors.

ATF4, as defined herein, means, unless expressly stated otherwise, human ATF4, having nucleic acid and amino acid sequences as set forth in GenBank Accession Numbers: NM001675, NM182810, NP001666, and NP877962 as well as nucleic acid and amino acid sequences having at least 95 percent homology thereto, as determined by standard homology-determining software such as, but not limited to, BLAST or FASTA. Also encompassed in the definition of ATF4 are nucleic acid molecules which hybridize with a nucleic acid having a sequence as set forth in GenBank Accession Nos. NM001675 and NM182810 under stringent hybridization conditions, such as hybridization in 0.5 M NaHPO₄, 7 percent sodium dodecyl sulfate (“SDS”), 1 mM ethylenediamine tetraacetic acid (“EDTA”) at 65° C., and washing in 0.1×SSC/0.1 percent SDS at 68° C. (Ausubel et al., 1989, Current Protocols in Molecular Biology, Vol. I, Green Publishing Associates, Inc., and John Wiley & Sons, Inc. New York, at p. 2.10.3), as well as the proteins encoded by such hybridizing nucleic acid molecules.

In one set of non-limiting embodiments, the anti-ATF4 agent may be a nucleic acid, such as a siRNA, an antisense nucleic acid, a ribozyme, or a DNA-zyme. Such nucleic acids may comprise one or more region which is at least about 80, 85, 90 or 95 percent homologous to ATF4 (in the case of antisense nucleic acid, homologous to the non-coding strand having a sequence which is the complement of the coding sequence) (as determined by standard homology-determining software), and, when introduced into a cell expressing ATF4, inhibit expression of ATF4.

In certain non-limiting embodiments of the invention, dsRNA-mediated interference (RNAi or siRNA) may be used to inhibit the expression of the ATF4 gene (see, for example, C. P. Hunter, Current Biology, 1999, 9:R440-442; Hamilton et al., 1999, Science 286:950-952; and S. W. Ding, Current Opinions in Biotechnology, 2000, 11:152-156). siRNA typically comprises a polynucleotide sequence identical or homologous to a target gene (or fragment thereof) linked directly, or indirectly, to a polynucleotide sequence complementary to the sequence of the target gene (or fragment thereof). The dsRNA may comprise a polynucleotide linker sequence of sufficient length to allow for the two polynucleotide sequences to fold over and hybridize to each other; however, a linker sequence is not necessary. The linker sequence is designed to separate the antisense and sense strands of siRNA sufficiently as to limit the effects of steric hindrance and allow for the formation of dsRNA molecules and should not hybridize with sequences within the hybridizing portions of the dsRNA molecule. Accordingly, one method for inhibiting ATF4 expression comprises the use of (siRNA) comprising polynucleotide sequences identical or homologous to the ATF4 gene.

RNA containing a nucleotide sequence identical to a fragment of the target gene is preferred for inhibition; however, RNA sequences with insertions, deletions, and point mutations relative to the target sequence can also be used for inhibition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

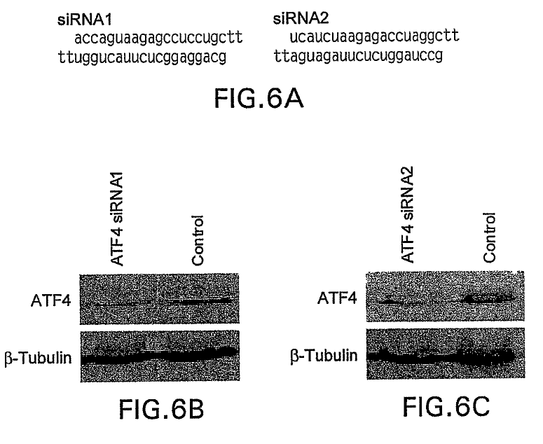

Preferably, siRNA is targeted to a polynucleotide sequence of the ATF4 gene. Preferred siRNA molecules of the instant invention are highly homologous or identical to the corresponding regions of the ATF4 gene. The homology may be greater than 70%, preferably greater than 80%, more preferably greater than 90% and is most preferably greater than 95%. Specific non-limiting embodiments of siRNAs that may be used to inhibit ATF4 expression are siRNA1 and siRNA2, as depicted in FIG. 6A and having SEQ ID NOS: 5 and 6, respectively.

In certain other embodiments, the anti-ATF4 nucleic acid agent is an antisense nucleic acid. Preferably, the antisense oligonucleotide sequence is at least six nucleotides in length, but can be up to about 50 nucleotides long. Longer sequences can also be used. The antisense oligonucleotides of the invention may be DNA, RNA, or any modifications or combinations thereof. As an example of the modifications that the oligonucleotides may contain, inter-nucleotide linkages other than phosphodiester bonds, such as phosphorothioate, methylphosphonate, methylphosphodiester, phosphorodithioate, phosphoramidate, phosphotriester, or phosphate ester linkages (Uhlman et al., 1990, Chem. Rev. 90(4):544-584; Tidd 1990, Anticancer Research 10(5A):1169-1182), may be present in the oligonucleotides, resulting in their increased stability. Oligonucleotide stability may also be increased by incorporating 3′-deoxythymidine or 2′-substituted nucleotides (substituted with, e.g., alkyl groups) into the oligonucleotides during synthesis, by providing the oligonucleotides as phenylisourea derivatives, or by having other molecules, such as aminoacridine or poly-lysine, linked to the 3′ ends of the oligonucleotides (see, e.g., Tidd 1990, Anticancer Research 10(5A): 1169-1182). Modifications of the RNA and/or DNA nucleotides comprising the oligonucleotides of the invention may be present throughout the oligonucleotide, or in selected regions of the oligonucleotide, e.g., the 5′ and/or 3′ ends. The antisense oligonucleotides may also be modified so as to increase their ability to penetrate the target tissue by, e.g., coupling the oligonucleotides to lipophilic compounds. The antisense oligonucleotides of the invention can be made by any method known in the art, including standard chemical synthesis, ligation of constituent oligonucleotides, and transcription of DNA encoding the oligonucleotides, as described below. Precise complementarity is not required for successful duplex formation between an antisense molecule and the complementary coding sequence of the ATF4 gene. Antisense molecules which comprise, for example, 2, 3, 4, or 5 or more stretches of contiguous nucleotides which are precisely complementary to a portion of a coding sequence of the ATF4 gene, each separated by a stretch of contiguous nucleotides which are not complementary to adjacent coding sequences, can provide targeting specificity for mRNA of the ATF4 gene. Preferably, each stretch of contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and the ATF4 gene untranslated or coding sequence.

Ribozymes, antisense polynucleotides, and siRNA molecules may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands); the promoters may be known inducible promoters such as baculovirus. The RNA strands may or may not be polyadenylated; the RNA strands may or may not be capable of being translated into a polypeptide by a cell's translational apparatus.

RNA may also be chemically or enzymatically synthesized by manual or automated reactions. The RNA may be synthesized by a cellular RNA polymerase or a bacteriophage RNA polymerase (e.g., T3, T7, SP6). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no, or a minimum of, purification to avoid losses due to sample processing. The RNA may be dried for storage or dissolved in an aqueous solution. The solution may contain buffers or salts to promote annealing, and/or stabilization of the duplex strands.

In other non-limiting embodiments, the anti-ATF4 agent may be an immunoglobulin molecule or a fragment thereof, such as, but not limited to, an Fv fragment, Fab fragment, F(ab)₂ fragment, or may be a single-chain antibody.

In further embodiments, the anti-ATF4 agent may be identified by a method identified by screening as discussed below in Section 5.4.

### 5.2 Methods of Diagnosing Alzheimer's Disease or Other ATF4-Related Disorders

In non-limiting embodiments, the present invention provides for methods of diagnosing AD or other neurological disorders, such as neurodegenerative disorders, associated with increased ATF4 by detecting elevated levels of ATF4 in a sample collected from a patient. In non-limiting embodiments, ATF4 protein expression may be detected and measured using an anti-ATF4 antibody, for example in the context of antiserum as prepared in Section 11, below. In specific non-limiting embodiments, the present invention provides for an antibody that binds peptide GLLDDYLEVAKHFKPHGFSSC (SEQ ID NO:7), and for an antibody that binds peptide FAPLVQETNKQPPQTVNPIGC (SEQ ID NO:8); either such antibody may be monoclonal or originate from a polyclonal antiserum.

In other non-limiting embodiments, the level of ATF4 expression may be assessed by measuring the level of ATF4 mRNA, where an increase in ATF4 mRNA correlates with an increase in ATF4 protein. A nucleic acid probe, designed using the nucleic acid sequence of ATF4, may be prepared using methods known in the art.

The patient sample may be a sample of brain tissue (see, for example, Warren, J. et al., 2005, Brain 128: 2016-2025), or, alternatively, a sample of cerebrospinal fluid, blood, etc.

In the case of AD, determining that the level of ATF4 is increased by a factor of at least about 1.5 relative to a suitable control (e.g., from an age-matched and optionally gender-matched individual) is supportive of a diagnosis of AD.

### 5.3 Methods of Treating Alzheimer's Disease or Other ATF4-Related Disorders

In non-limiting embodiments, the present invention provides for methods of treating Alzheimer's Disease or other neurological disorders associated with increased ATF4 expression, comprising administering, to a subject in need of such treatment, an anti-ATF4 agent. In particular non-limiting embodiments, the neurological disorder is a neurodegenerative disorder.

In one set of embodiments, the anti-ATF-4 agent which may be administered may be a nucleic acid molecule, such as an siRNA, antisense nucleic acid, ribozyme, or DNA-zyme. Specific non-limiting examples of siRNAs which have been shown to be effective in inhibiting ATF4 expression in cells in culture are siRNA1 and siRNA2, as depicted in FIG. 6A, having SEQ ID NO:5 and 6. Such nucleic acid molecules may be administered

Ribozymes, antisense molecules, or siRNA can be introduced into cells as part of a DNA construct, as is known in the art. The DNA construct can also include transcriptional regulatory elements, such as a promoter element (such as, not by way of limitation, neuron or brain specific promoter, see below) an enhancer or UAS element, and a transcriptional terminator signal, for controlling the transcription of the ribozyme in the cells. Mechanical methods, such as microinjection, liposome-mediated transfection, electroporation, or calcium phosphate precipitation, can be used to introduce such DNA constructs into cells whose division it is desired to decrease, as described above. Alternatively, if it is desired that the DNA construct be stably retained by the cells, the DNA construct can be supplied on a plasmid and maintained as a separate element or integrated into the genome of the cells, as is known in the art.

Any of antisense nucleic acid, siRNA, ribozyme or DNA-zyme may optionally be comprised in a microstructure such as a liposome or microsphere. A composition comprising the agent may further comprise a permeability-enhancing agent such as dimethylsulfoxide, lipofectamine, oligofectamine, nanoparticles, and/or cyclofectin.

In further non-limiting embodiments, the anti-ATF4 agent may be a single chain antibody. Such single-chain antibody may be introduced into cells of the nervous system by introducing a nucleic acid encoding the single-chain antibody, in expressible form (for example, operably linked to a promoter element which is active in the central nervous system, such as a housekeeping (constitutively active) promoter or a neuron and/or CNS tissue-specific promoter, such as the myelin basic protein promoter, neuron specific enolase promoter, astrocyte specific glial fibrillary acidic protein (GFAP) promoter, neurofilament promoter, neuron specific platelet-derived growth factor β-chain (PDGE-β) promoter, human cytomegaalovirus (HCMV) promoter, synapsin-1 (Syn1) promoter, tubulin-α1 (Tα1) promoter, and PRSx8 promoter. Such nucleic acid may be introduced, as naked DNA or comprised in a suitable vector, into the brain tissue by local application or injection or by administration into the cerebrospinal fluid.

In further embodiments, the anti-ATF4 agent may be an agent identified by screening methods set forth in the following section.

### 5.4 Drug Discovery Methods

In still further embodiments, the present invention provides for drug discovery methods that identify agents that may be useful in the treatment of Alzheimer's Disease or other neurological disorders associated with increased ATF4 expression by screening for agents that inhibit ATF4 expression.

In a first series of embodiments, the present invention provides for the use of a cell line that expresses ATF4 in drug discovery methods. Accordingly, the present invention provides for a method of identifying an agent useful in treating AD or other neurological disorder associated with increased ATF4 expression comprising culturing a cell line which expresses detectable levels of ATF4 in the presence of a test agent, and then comparing the level of ATF4 in the cell line exposed to test agent to the level of ATF4 expressed in the cell line in the absence of test agent, wherein a decrease of ATF4 level in the presence of test agent is consistent with utility of the agent for treating AD or said other neurological disorder. In specific, non-limiting embodiments, the cell is a human cell, for example a SW13 adrenal carcinoma cell, a human embryonic kidney 293 cell or a HeLa cell. In particular, non-limiting embodiments, the cell is a non-human cell, in which case the ATF4 measured is either human ATF4 which has been introduced into the cell, or ATF4 of the species of the non-human cell.

In a second series of embodiments, the present invention provides for the use of a cell line, exposed to increased amyloid beta (e.g., Aβ42 peptide), in drug discovery methods. Accordingly, the present invention provides for a method of identifying an agent useful in treating AD comprising culturing a cell line in the presence of amyloid beta peptide, where cells of the culture, in the presence of amyloid beta peptide, exhibit elevated levels of ATF4, adding a test agent to the amyloid beta-exposed cell culture, and then comparing the level of ATF4 in the cell line exposed to amyloid beta peptide and test agent to the level of ATF4 expressed in the cell line in the presence of amyloid beta peptide and in the absence of test agent, wherein a decrease of ATF4 level in the presence of amyloid beta peptide and test agent relative to the ATF4 level in the presence of amyloid beta peptide and in the absence of test agent is consistent with utility of the agent for treating AD. In specific, non-limiting embodiments, the cell is a human cell. In particular, non-limiting embodiments, the cell is a non-human cell, in which case the ATF4 measured is either human ATF4 which has been introduced into the cell, or ATF4 of the species of the non-human cell. In this and other embodiments, the level of ATF4 may be measured by any method known in the art, including, but not limited to, PCR analysis, Northern blot, Western blot, immunohistochemistry, etc.

In a third series of embodiments, the present invention provides for the use of a cell having endogenously increased amyloid beta (e.g., Aβ42 peptide), in drug discovery methods. Accordingly, the present invention provides for a method of identifying an agent useful in treating AD comprising providing a cell having endogenously increased amyloid beta peptide and exposing the cell to a test agent, and then comparing the level of ATF4 in the cell exposed to test agent to the level of ATF4 expressed in a comparable cell in the absence of test agent, wherein a decrease of ATF4 level in the presence of test agent is consistent with utility of the agent for treating AD. The cell may be an isolated cell (for example, as in a cultured cell line) or a cell which forms part of a tissue and/or a cell which forms part of an animal. In specific, non-limiting embodiments, the cell is a human cell. In particular, non-limiting embodiments, the cell is a non-human cell, in which case the ATF4 measured is either human ATF4 which has been introduced into the cell, or ATF4 of the species of the non-human cell. As one specific, non-limiting example, the cell is a cell of a J20 transgenic mouse and the ATF4 is murine ATF4, and a comparable cell may be a cell of the same type in an animal not exposed to test agent.

In a fourth series of embodiments, the present invention provides for a method of identifying an agent useful in treating Alzheimer's Disease or another neurological disorder associated with increased ATF4 expression comprising:
(i) providing cells which express detectable levels of ATF4 and contain an expression construct comprising a reporter gene operably linked to a ATF4-responsive promoter,
(ii) exposing said cells to a test agent;
(ii) measuring the level of reporter gene expressed in the cells exposed to the test agent; and
(iii) comparing the level of reporter gene expression in the cells exposed to the test agent to the level of reporter gene expression in control cells (cells the same as or otherwise having all the relevant features of the cells exposed to test agent, including, but not limited to, cells obtained from the same source maintained in parallel conditions (e.g., a parallel culture, a different transgenic animal) or a cell sample collected prior to exposure to the test agent) not exposed to the test agent; wherein an increase in reporter gene expression in the cells exposed to the test agent relative to the control cells is consistent with the utility of the test agent for treating Alzheimer's disease or said other neurological disorder.

According to this fourth embodiment, examples of suitable cell lines include HEK 293 cells, PC12 cells and SH-SY5Y cells. The reporter gene may encode a detectable product which may be its mRNA transcript or, preferably, a reporter protein, which may be any protein known in the art, including but not limited to a green fluorescent protein (GFP), enhanced green fluorescent protein, red fluorescent protein (RFP), yellow fluorescent protein (YFP), blue fluorescent protein (BFP), luciferase, or beta-galactosidase, or known variants thereof. In one specific, non-limiting embodiment, the reporter protein may be d2EGFP (Clontech), a destabilized variant of Enhanced GFP, wherein residues 422-461 of mouse ornithine decarboxylase (MODC) are fused to the C-terminus of EGFP; since this region of MODC contains a PEST sequence, the protein is targeted for rapid turnover. The excitation and emission maxima of d2EGFP are 488 nm and 509 nm, respectively.

According to this fourth series of embodiments, the ATF4-responsive promoter may be any promoter which is ATF4-responsive, including promoters that comprise at least one (preferably more than one, for example, but not by way of limitation, two or three) cAMP Responsive Element (CRE element), for example but not limited to an asparagine synthase promoter, C/EBP homologous protein (CHOP) promoter, the insulin gene promoter (Hay et al., 2007, Biochim. Biophys. Acta. 1769(2):79-91), the CD14 gene promoter (Moeenrezakhanlou et al., 2007, J. Leukocyte. Biol. Feb. 27, 2007 (e-pub),) or the ERG-1 gene promoter (Kang et al., 2007, Am. J. Physiol. Endocrinol. Metab. 292(1):E215-22). In one specific, non-limiting embodiment, the ATF4-responsive promoter may be the promoter of the pCRE-luc vector sold by Clontech (Catalog # 631911), which drives a firefly luciferase reporter, the expression of which may be monitored using a luminometer, according to standard techniques. Interaction of ATF4 with a promoter comprising one or more CRE element would be expected to decrease (inhibit) promoter activity. In a specific, non-limiting embodiment, the promoter may comprise three copies of the CRE-binding sequence fused to a TATA-like promoter (P_{TAL}) region from the Herpes simplex virus thymidine kinase (HSV-TK) promoter.

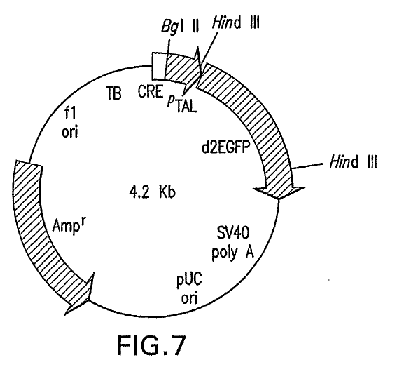

In one specific, non-limiting example of this fourth series of embodiments, a reporter construct which may be introduced into a neuronal cell, such as a PC12 cell, may be a pCRE-d2EGFP construct (Clontech, but believed to be a discontinued product). A diagram of pCRE-d2EGFP (Clontech) is shown in FIG. 7. In pCRE-d2EGFP, the reporter protein coding sequence is followed by the SV40 late polyadenylation signal. A synthetic transcription blocker (TB) having adjacent polyadenylation and transcription pause sites is positioned upstream of the enhancer to reduce background transcription. The construct further comprises an fl origin for single-stranded DNA synthesis, a pUC origin of replication, and an ampicillin resistance gene.

In this fourth series of non-limiting embodiments, a construct comprising a gene encoding a reporter protein, operably linked to an ATF4-responsive promoter, may be introduced into a neuronal cell, such as a PC12 cell, optionally concurrently with a selection marker. Stable transfectants may be selected. To identify cell colonies that are responsive to CREB activation, cells may be treated with Nerve Growth Factor (NGF) or forskolin, and the expression level of reporter protein may be evaluated. Analogous methods may be used to produce a transgenic animal.

As a specific, non-limiting example within the fourth series of embodiments, a CRE-d2EGFP-transfected, stable PC12 cell line may be prepared, and tested to determine whether amyloid beta (Aβ42) peptide stimulates expression of ATF4 and inhibits expression of d2EGFP in NGF-treated CRE-d2EGFP PC12 cells. Increased expression of ATF4 and decreased expression of d2EGFP in an NGF-treated CRE-d2EGFP PCI2 cell line is consistent with suitability of the cell line for drug discovery according to the invention. The cell line may also be tested to determine whether knock-down of ATF4 by siRNA in amyloid beta (e.g., Aβ42 peptide)-treated cells results in increase of d2EGFP expression, where such increased d2EGFP expression is consistent with suitability of the cell line for drug discovery according to the invention. The ability of a test agent to increase reporter protein (e.g., d2EGFP) expression in a cell according to this fourth series of embodiments is consistent with utility of the agent for treating AD.

### 6. EXAMPLE

### ATF4 is Increased in PC12 Cells Exposed to Amyloid Beta

Micro-Serial Analysis of Gene Expression (“SAGE”) was carried out on neuronally differentiated PC12 cells exposed to 10 micromolar Aβ42 peptide for three hours. The SAGE-detected genes were compared to those in a pre-existing SAGE library of control neuronally-differentiated cells. Among the genes with changed expression levels was ATF4, which showed a 10.5-fold elevation in response to Aβ42. Subsequent studies established that this was also accompanied by an elevation of ATF4 protein and that similar changes in protein expression occur in cultured hippocampal neurons exposed to Aβ42.

### 7. EXAMPLE

### ATF4 is Increased in an Animal Model of Alzheimer's Disease

SAGE was performed on 6 month-old J20 mice (an Alzheimer's Disease animal model) and control littermates. J20 transgenic mice carry a mutant human APP transgene with both the Swedish (K670N, M67L) and Indiana (V717F) familial AD mutations (Mucke et al., J. Neurosco. 20(11):4050-4058). As a result, J20 mice express high levels of Aβ and show progressive behavioral and anatomical degeneration characteristic of human AD. SAGE libraries were constructed using RNA collected from pooled hippocampi of three J20 male mice and three matched controls. After analyzing more that 100,000 SAGE tags, the expression of murine ATF4 was found to be upregulated in J20 mouse hippocampi.

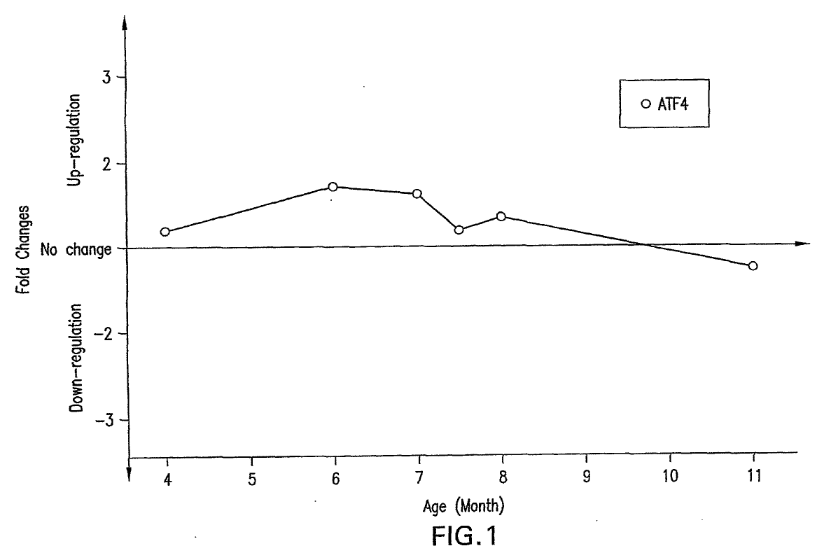

To further explore the difference in expression levels between J20 and control mice, real-time RT-PCRs were performed using total RNAs isolated from hippocampi of J20 and matched control mice at different ages. PCRs were conducted using the Brilliant SYBR Green fluorescent dye (Invitrogen), the OmniMix™ HS PCR beads (taKaRa Bio Inc., Otsu, Shiga, Japan), and the Smart Cycler II thermal cycler (Cepheid, Sunnyvale, Calif., USA). The sequences of mouse ATF4 PCR primers were 5′-gaagcctgactctgctgctt-3′ (SEQ ID NO:1) and 5′-gtggctgctgtcttgttttg-3′ (SEQ ID NO:2). The PCR condition was 1 cycle of 5 minutes at 95° C. and then 40 cycles of 15 seconds (s) at 95° C., 30 s at 62° C., 30 s at 72° C., and 20 s at 88° C., for measuring fluorescence intensity. GAPDH was used as the internal control for normalization. The relative abundance of murine ATF4 in J20 to control was calculated by the Delta-delta method: relative abundance=2^{-ΔΔCt}, where ΔΔCt=[(Ct:ATF4,J20−Ct:GAPDH,J20)−(Ct:ATF4,control−Ct:GAPDH,control)]; Ct=threshold cycle. The result is illustrated in FIG. 1, which shows that increased ATF4 expression was found in young animals and then appeared to decrease to normal in older animals.

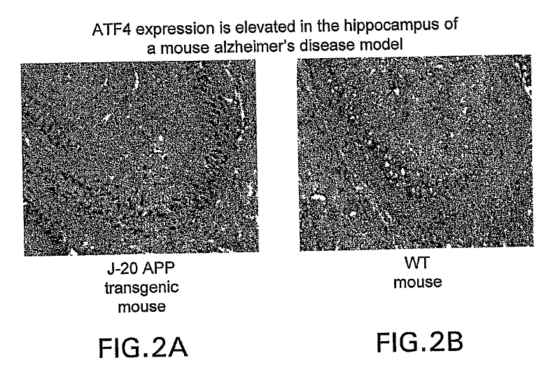

FIG. 2A-B presents the results of immunostaining hippocampal sections from an adult J-20 APP mouse, relative to wild-type, with anti-ATF4 antiserum. Increased staining of the J-20 APP hippocampal tissue indicates that ATF4 expression is elevated in this murine model of Alzheimer's disease.

### 8. EXAMPLE

### Comparison of ATF4 mRNA Levels Among Normal Individuals and Alzheimer Disease Patients

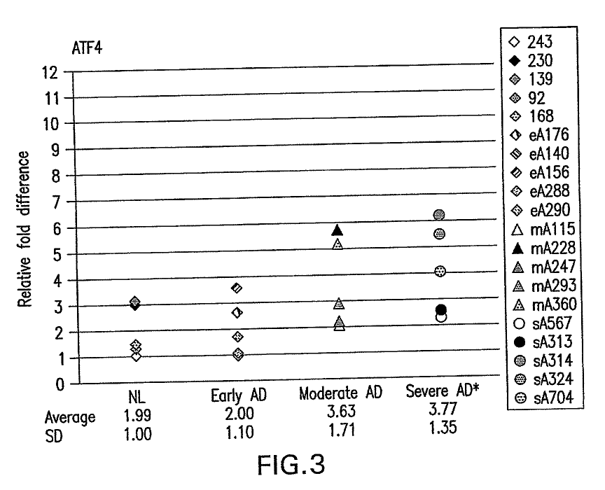

Real-time RT-PCRs were performed using total RNAs isolated from entorhinal cortices of five normal individuals and fifteen AD patients who were at different disease stages. Entorhinal cortex was chosen for this study because AD neuropathology is thought to begin in this region, progresses to the hippocampus, and finally spreads throughout the limbic system and neocortex. PCR conditions were similar to those used for measuring the mouse ATF4 levels as described in the previous example section, except that the annealing temperature and the fluorescence reading temperatures were set at 68° and 89° C., respectively. The sequences of human ATF4 PCR primers were 5′-gttgggtatagatgacctggaaac-3′ (SEQ ID NO:3) and 5′-cccagctctaaactaaaggaatga-3′ (SEQ ID NO:4). Relative amounts of ATF4 transcripts of each individual are plotted on a graph in FIG. 3. Although there were noticeable differences in ATF4 levels between individuals even within the same group, on the whole, statistically significant up-regulation of ATF4 was found in severe stage AD patients (P<0.05 versus normal).

### 9. EXAMPLE

### Immunohistochemical Analysis of ATF4 Protein in Post-Mortem Tissues from Normal Individuals and AD Patients

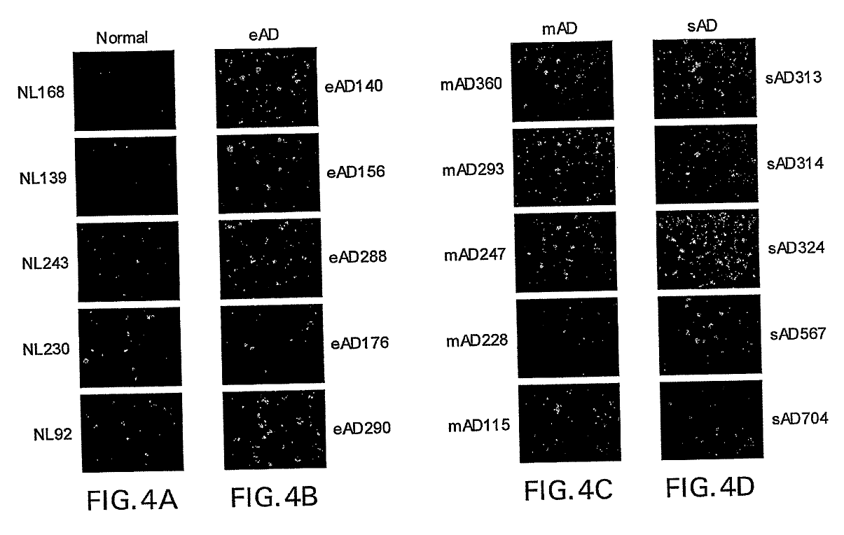

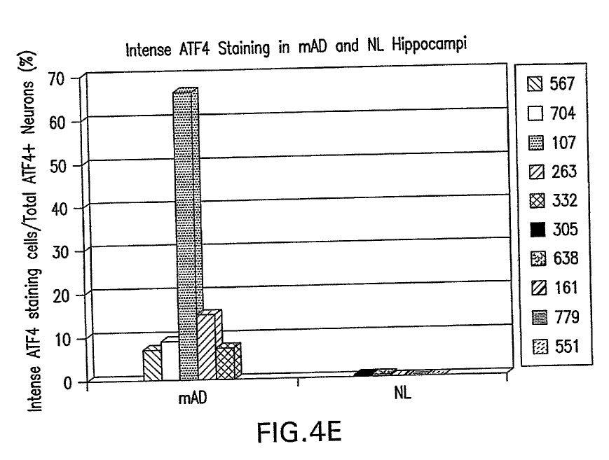

To study the alterations of ATF4 in AD at the protein level, polygonal antibodies were raised against ATF4 peptides. Two antibodies, #18 and #21, were found to be useful for Western blot analysis and immunohistochemistry studies. An affinity-purified antiserum proved to be highly specific for recognition of ATF4. Immunostainings for ATF4 were performed on entorhinal cortex frozen sections of post-mortem control and AD brains (FIG. 4A-E).

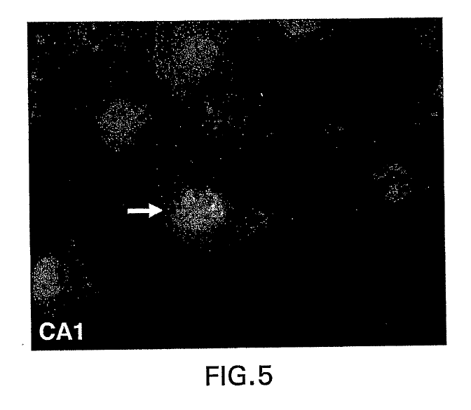

As illustrated in FIG. 4A-E, even though AD brains appeared to show some elevated expression of the protein, the difference between AD and normal brains was not very dramatic. Therefore, the ATF4 staining was studied in more detail, and other regions of the post-mortem brains were analyzed. It was found that ATF4 protein levels were dramatically up-regulated in some but not all neurons in affected areas of AD brains (FIG. 5). In the CA1 region of the hippocampus, 5-15 percent of neurons showed extremely intense nuclear staining for ATF4. Similar high-intensity ATF4 staining was not observed in normal brains. One interpretation of this observation is that those neurons with high levels of ATF4 protein are undergoing pathological changes.

### 10. EXAMPLE

### Reduction of ATF4 Expression by siRNA

siRNAs were developed complementary to ATF4 to “knock-down” the expression of ATF4 in human cells. Two double stranded siRNAs (siRNA1 and siRNA2, having SEQ ID NOS:5 and 6, respectively, as shown in FIG. 6A) were prepared by annealing two sets of complementary RNAs. Efficacy was tested by transient transfection of SW13 cells, which are adrenal carcinoma cells that express ATF4. The amount of endogenous ATF4 in the cells was measured by Western blotting using #18 antibody. As illustrated in FIGS. 6B and C, both siRNAs can significantly knock-down the expression of ATF4.

### 11. EXAMPLE

### Anti-ATF4 Antibodies

Antibodies #18 and #21 as discussed above, polyclonal antibodies raised in rabbits, were prepared as follows. Synthetic peptides GLLDDYLEVAKHFKPHGFSSC (SEQ ID NO:7) and FAPLVQETNKQPPQTVNPIGC (SEQ ID NO:8) were used to for immunizations resulting in antibodies #18 and #21, respectively. 1.5 mg of each peptide was conjugated with 5 mg of KLH (keyhole limpet hemocyanin) for immunization.
Day 0: Injected 200 ug of conjugated peptide (CP) in Complete Freund's Adjuvant (CFA) intradermally.
Day 14: Injected 100 ug of CP intradermally in Incomplete Freund's Adjuvant (IFA).
Day 28: Injected 100 ug of CP intradermally with IFA.
Day 42: Small bleed for testing.
Day 56: Injected 50 ug of CP subcutaneously with IFA.
Day 70: Small bleed for testing.
Afterwards, 50 ug of CP with IFA was injected subcutaneously for boosting and production bleeds were collected every two weeks.

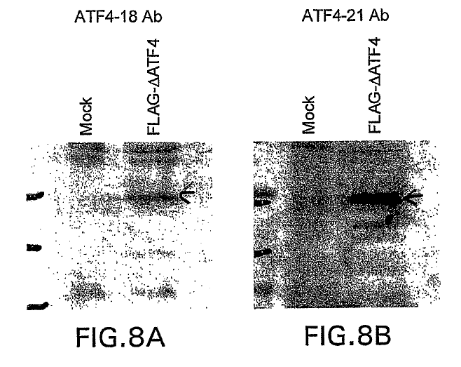

To examine the specificity of the antibodies, an expression plasmid (pFLAG-DATF4) containing a FLAG-tagged truncated ATF4 cDNA was constructed. Transient transfections were performed using pFLAG-DATF4 on SW13 cells. 48 hours post-transfection, cell lysates were collected for Western blot analyses using antibodies #18 and #21, the results of which are shown in FIG. 8A-B. Both antibodies were then affinity purified and the specificity of antibody #18 was further examined.

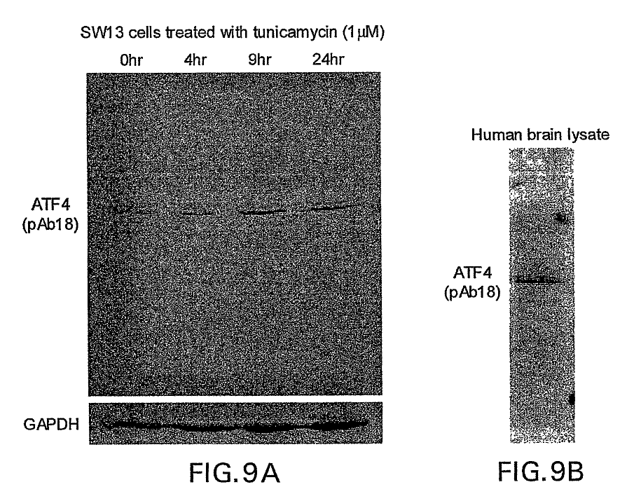

Because ATF4 has been shown to be up-regulated in cells that were treated with tunicamycin, SW13 cells were treated with 1 micromolar tunicamycin. Cell lysates were collected for Western blotting at various time points. As shown in FIG. 9A, a single specific band was detected in the cell lysates. The intensity of the band was higher in lysates collected 9 and 24 hours after tunicamycin treatment, suggesting that the detected band corresponded to ATF4 protein. More importantly, the band migrated at ˜38 kDa, the size predicted for human ATF4. Purified Ab #18 was also tested on crude human brain lysates and detected one specific ˜38 kDa band (FIG. 9B).

### 12. EXAMPLE

### Effect of Depletion of ATF4 on Neurite Growth

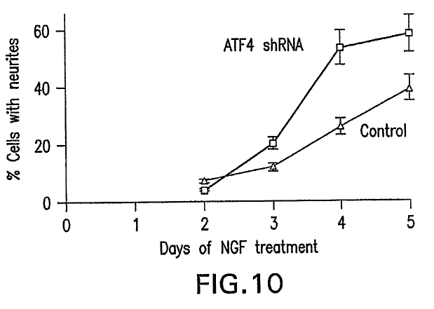

Cultured PC12 cells were transfected with either eGFP (a marker for engineered cells) alone (“control”) or eGFP+ATF4-specific interfering RNA (“ATF4-shRNA”). The cells were then exposed to 50 ng/ml of human recombinant nerve growth factor (“NGF”) and then, after various times of treatment (as indicated in FIG. 10), scored for the presence of neurites.

As shown in FIG. 10, knock-down of ATF4 expression with siRNA resulted in an increased percentage of cells with neurites relative to transfected control cells which did not receive siRNA. These data indicate that endogenous ATF4 represses the capacity of cells to produce neurites in response to trophic stimulation. Therefore, the increase in ATF4 levels observed in Alzheimer's disease can result in decreased neurite outgrowth with consequent cognitive defects. Decreasing ATF4 expression can promote neurite outgrowth and potentially reverse such effects.

In related experiments, PC12 cells were again transfected either eGFP (a marker for engineered cells) alone (“control”) or eGFP+ATF4-specific interfering RNA (“ATF4-shRNA”) and then the cultures were treated with 50 ng/ml of human recombinant NGF. Five days later, random fields of the cultures were photographed and assessed for average neurite length (±SEM) of transfected cells. 50 control and 25 ATF4 siRNA cells were evaluated.

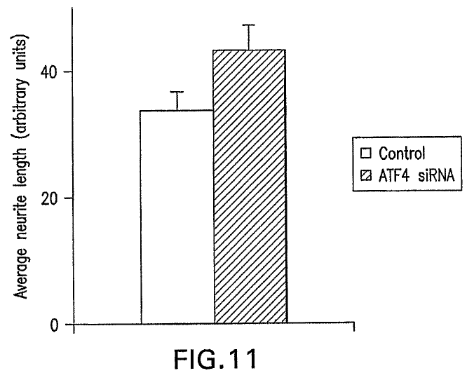

The results, shown in FIG. 11, show that average neurite length was observed to be increased in siRNA-receiving cells. These findings indicate that endogenous ATF4 reduces capacity for neurite outgrowth and that reducing endogenous ATF4 levels can promote neurite outgrowth.

Various references are cited herein, the contents of which are hereby incorporated by reference herein in their entireties.

We claim:

1. A method of treating a neurodegenerative disorder associated with increased ATF4 expression, comprising administering, to a subject in need of such treatment, an anti-ATF4 agent in an amount effective to decrease ATF4 expression and/or activity.

2. The method of claim 1, wherein the neurodegenerative disorder is Alzheimer's Disease.

3. The method of claim 1 or claim 2, wherein the anti-ATF4 agent is a siRNA.

4. The method of claim 1 or claim 2, wherein the anti-ATF4 agent is an antisense nucleic acid.

5. The method of claim 1 or claim 2, wherein the anti-ATF4 agent is a ribozyme.

6. The method of claim 1 or claim 2, wherein the anti-ATF4 agent is a DNA-zyme.

7. The method of claim 1 or claim 2, wherein the anti-ATF4 agent is a single chain antibody.

8. The method of claim 7, wherein the single chain antibody is administered by introducing, into a cell of the subject, a nucleic acid encoding the single chain antibody, in expressible form.

9. A method of diagnosing, in a subject, a neurodegenerative disorder associated with an increased level of ATF4, comprising detecting the level of ATF4 expression in a sample from the subject, and comparing the level to a control value, where an increase in ATF4 expression supports a diagnosis of the neurodegenerative disorder.

10. The method of claim 9, wherein the neurodegenerative disorder is Alzheimer's Disease.

11. The method of claim 9, wherein the ATF4 level is measured using an antibody.

12. The method of claim 9, wherein the ATF4 level is measured by detecting ATF4 mRNA using a nucleic acid probe.

13. An isolated siRNA molecule having SEQ ID NO:5.

14. An isolated siRNA molecule having SEQ ID NO:6.

15. A purified antibody that specifically binds a peptide GLLDDYLEVAKHFKPHGFSSC (SEQ ID NO:7).

16. The antibody of claim 15 which originates in a polyclonal antiserum.

17. The antibody of claim 15 which is a monoclonal antibody.

18 A purified antibody that specifically binds peptide FAPLVQETNKQPPQTVNPIGC (SEQ ID NO:8).

19. The antibody of claim 18 which originates in a polyclonal antiserum.

20. The antibody of claim 18 which is a monoclonal antibody.

21. A method of identifying an agent useful in treating Alzheimer's Disease or another neurological disorder associated with increased ATF4 expression comprising culturing a cell line which expresses detectable levels of ATF4 in the presence of a test agent, and then comparing the level of ATF4 in the cell line exposed to test agent to the level of ATF4 expressed in the cell line in the absence of test agent, wherein a decrease of ATF4 level in the presence of test agent is consistent with utility of the test agent for treating Alzheimer's disease or said other neurological disorder.

22. The method of claim 21, where the cell is selected from the group consisting of a SW13 cell, a PC12 cell, or an SH-SY5Y cell.

23. A method of identifying an agent useful in treating Alzheimer's disease comprising culturing a cell line in the presence of amyloid beta peptide, where cells of the culture, in the presence of amyloid beta peptide, exhibit elevated levels of ATF4, adding a test agent to the amyloid beta-exposed cell culture, and then comparing the level of ATF4 in the cell line exposed to amyloid beta peptide and test agent to the level of ATF4 expressed in the cell line in the presence of amyloid beta peptide and in the absence of test agent, wherein a decrease of ATF4 level in the presence of amyloid beta peptide and test agent relative to the ATF4 level in the presence of amyloid beta peptide and in the absence of test agent is consistent with utility of the test agent for treating Alzheimer's disease.

24. A method of identifying an agent useful in treating Alzheimer's disease comprising providing a cell having endogenously increased amyloid beta peptide and exposing the cell to a test agent, and then comparing the level of ATF4 in the cell exposed to test agent to the level of ATF4 expressed in a comparable cell in the absence of test agent, wherein a decrease of ATF4 level in the presence of test agent is consistent with utility of the test agent for treating Alzheimer's disease.

25. The method of claim 24, wherein the cell is a cell of a J20 transgenic mouse and the ATF4 is murine ATF4, and a comparable cell may be a cell of the same type in a J20 transgenic animal not exposed to test agent.

26. A method of identifying an agent useful in treating Alzheimer's Disease or another neurological disorder associated with increased ATF4 expression comprising:
(i) providing cells which express detectable levels of ATF4 and contain an expression construct comprising a reporter gene operably linked to a ATF4-responsive promoter,
(ii) exposing said cells to a test agent;
(ii) measuring the level of reporter gene expressed in the cells exposed to the test agent; and
(iii) comparing the level of reporter gene expression in the cells exposed to the test agent to the level of reporter gene expression in control cells not exposed to the test agent;
wherein an increase in reporter gene expression in the cells exposed to the test agent relative to control cells is consistent with the utility of the test agent for treating Alzheimer's disease or said other neurological disorder.

27. The method of claim 26, wherein the ATF4-responsive promoter comprises at least one cAMP Responsive Element (CRE element).

28. The method of claim 27, wherein the ATF4-responsive promoter is the asparagine synthase promoter.

29. The method of claim 27, wherein the ATF4-responsive promoter is the C/EBP homologous protein (CHOP) promoter.

30. The method of claim 27, wherein the ATF4-responsive promoter comprises three copies of the CRE-binding sequence fused to a TATA-like promoter (P_{TAL}) region from the Herpes simplex virus thymidine kinase (HSV-TK) promoter.


SEQUENCE LISTING (8 sequences)

SEQ ID NO: 1 - DNA, 20 bp, Artificial sequence
  synthetic oligonucleotide
gaagcctgactctgctgctt

SEQ ID NO: 2 - DNA, 20 bp, Artificial sequence
  synthetic oligonucleotide
gtggctgctgtcttgttttg

SEQ ID NO: 3 - DNA, 24 bp, Artificial sequence
  synthetic oligonucleotide
gttgggtatagatgacctggaaac

SEQ ID NO: 4 - DNA, 24 bp, Artificial sequence
  synthetic oligonucleotide
cccagctctaaactaaaggaatga

SEQ ID NO: 5 - DNA, 42 bp, Artificial sequence
  synthetic oligonucleotide
accagnaagagccnccngcttttnggncanncncggaggacg

SEQ ID NO: 6 - DNA, 42 bp, Artificial sequence
  synthetic oligonucleotide
ncancnaagagaccnaggcttttagnaganncncngganccg

SEQ ID NO: 7 - protein, 21 aa, Artificial sequence
  synthetic peptide
GLLDDYLEVAKHFKPHGFSSC

SEQ ID NO: 8 - protein, 21 aa, Artificial sequence
  synthetic peptide
FAPLVQETNKQPPQTVNPIGC